(19) United States
(12) Patent Application Publication
(10) Pub. No.: US 2019/0022162 A1
(43) Pub. Date: Jan. 24, 2019

(54) COMBINATION ANTICANCER ENDOWED WITH ANTITUMOR ACTIVITY, COMPRISING ALKALOIDS OF CHELIDONIUM MAJUS

(71) Applicants: International Society for Drug Development S.R.L., Milano (IT); Advanced Molecular Biological Computation S.R.L., Pisa (IT)

(72) Inventors: Renata Batistoni, Asciano (IT); Anna Maria Paola Bianucci, Pisa (IT); Alessio Ferrari, Avezzano (IT); Pierluigi Madau, Sassari (IT); Giuseppe Lubinu, Sassari (IT); Silvia Marracci, Viareggio (IT); Marco Natali, Pietrasanta (IT); Guido Puricelli, Milano (IT)

(21) Appl. No.: 15/547,330

(22) PCT Filed: Jan. 29, 2016
(86) PCT No.: PCT/IB2016/050460

§ 371 Date: Jul. 28, 2017

(30) Foreign Application Priority Data
Jan. 29, 2015 (IT) GE2015A000010

(51) Int. Cl. A61K 36/66; A61K 31/495; A61K 31/4745; A61K 31/4741; A61K 31/395; A61K 31/7068

(52) U.S. Cl. CPC A61K 36/66; A61K 31/495; A61K 31/7068; A61K 31/4741; A61K 31/395; A61K 31/4745

(57) ABSTRACT

The invention relates to a combination having antitumour activity, wherein an agent having antineoplastic activity, such as gemcitabine or temozolomide, is associated with an alkaloid of Chelidonium majus (C. majus), such as berberine, chelidonine or protopine.

The present invention relates, in a general aspect thereof, to the treatment of tumour cells by means of compositions comprising alkaloids.

As it is known, the positive effects of this type of substances for treating cancerous cells have been studied for a number of applications, such as those described in International patent applications WO 2005/011698; WO1996/19226; WO1997/022201—“Procédé et système de . . . ”, wherein reference is made to the use of alkaloids of Vinca.

The aim of these studies is to find combinations of substances that will allow reducing the toxic effects of chemotherapeutic treatments for cancer pathologies.

In the above-mentioned patent applications the use of alkaloids of Vinca combined with oncolytic agents is described for the treatment of breast and prostate cancer, describe such as suramin (which is a salt of a polysulfonated naphtyl), also known as salt of Germanin, Mafuride and Antrypol.

The effects of the alkaloids of Vinca do not seem, however, to be particularly advantageous, since they can be replaced by other compounds such as, for example, estramustine, which is a derivative of estradiol, commercially available as a salt (estramustine phosphate sodium) and indicated for the palliative treatment of patients with metastases.

On the other hand, other alkaloids are also known in the prior art which have proven to exert antitumour activity: these include some alkaloids of Chelidonium majus (C. majus), to which the present invention relates.

C. majus is a herbaceous plant belonging to the poppy family, which grows spontaneously in Italy (commonly called chelidonium or celandine) and in other Mediterranean countries, as well as in many other places on the planet whose official properties, historically known are related to the presence of numerous active principles.

C. majus is also used in herbal medicine, homeopathy and phytotherapy for the treatment of spastic disorders of the biliar ducts and of the gastrointestinal tract, in preparations endowed with analgesic and sedative actions upon the central nervous system.

As can be inferred from such a broad range of applications, the active principles contained in C. majus are numerous and are not limited to alkaloids, since they also include other classes of compounds (vitamines, flavonoids, proteins, etc.), for which reference should be made to the existing literature in the field.

However, the current use of C. majus and its alkaloids is mainly directed towards the treatment of affections or pathologies of the skin or of the mucosae (dermatitis, verrucae, lesions, cicatrices, benign skin tumours, etc.); in this regard, dermatological uses of alkaloids of C. majus (as keratolytic agents) are already known from International patent application WO 2009/112226 and American patent application US 2006/0045930.

In addition, two International patent applications WO 2013/084163 and WO 2013/084162 describe the dermatological effects of particular extracts of C. majus and of alkaloids contained in this plant; they suggest that some of the most abundant alkaloids present in C. majus can modulate various cellular functions, in some cases increasing cell viability and proliferation. These documents also describe antifibrotic and anti-inflammatory properties possessed by some alkaloids of C. majus.

However the use of alkaloids of C. majus for antitumour applications has not been explored in depth yet, in that, to the Applicants' knowledge, most of the existing scientific information and publications are not related to consolidated therapeutic use of individual components of C. majus (mainly alkaloids, even though other components of this plants are also mentioned in the scientific literature) or extracts thereof.

Thus, for example, the use of a specific semi-synthetic preparation, commercially called “Ukrain™”, is known for therapeutical purposes, which contains, besides other components, also a mixture of alkaloids of C. majus that has not yet been exactly quantified. This preparation, however, which is currently in use in a number of countries, has never been approved by health authorities in regulatory USA and Europe.

Many studies exist on the effects of individual alkaloids contained in C. majus upon particular cancer cell lines, as well as studies on the effects of extracts of C. majus, conducted both in vitro and in vivo, aimed at identifying extracts potentially having antitumour properties, but the descriptions of the results do not contain any significant elements that might anticipate those characterizing the studies considered herein.

Indeed, the data reported in the scientific literature and in patents or patent applications in regard to the alkaloids of C. majus describe aspects which are different from those that this study intends to claim. For a better understanding of the work taken into consideration herein, a few documents are listed below, which, although they make mention of the molecules of interest, cannot however be considered as state of technique (prior art) because, as will be further explained below, they focus on aspects other than those highlighted in this work.

The effects of Ukrain and its combinations with known drugs (clinical aspects and in vitro studies) have been explored in the past and also in more recent times.

As far as clinical studies on Ukrain are concerned, two articles can be mentioned by way of example which are somehow related to what will be described herein, although the reported data essentially refer to aspects that are different from those highlighted in this application.

Staniszewski et al [1992] describe a clinical study on patients affected by previously untreated lung cancer, essentially focusing on the effect produced by Ukrain on a sub-population of lymphocytes.

In Gansauge et al. [2002] there is reported a clinical study on patients suffering from pancreatic cancer. In this document, Ukrain is erroneously defined (as in other documents) as a preparation containing a molecule of thiotepa that coordinates 3 molecules of chelidonine. This initially proposed structure was then proven wrong by Habermehl et al. [2006] and Voloshchuk et al. [2006].

In Gansauge et al. [2007] other clinical data are reported about beneficial effects of Ukrain associated with gemcitabine in patients suffering from pancreatic cancer; these results, however, did not lead to the inclusion of Ukrain among the drugs approved by the FDA or by many European regulatory authorities, presumably because of lack of accuracy in the collection and processing of the clinical data.

This justifies the research conducted by the present Applicants, aimed at identifying one or more components of Ukrain that are most responsible for such beneficial effects, and which have been described hitherto only in general terms in the literature about Ukrain. In this connection specific ranges are indicated for the quantities of the agents that need to be present around the cell, in order to provide the desired effect.

Some in vitro studies also exist on the effects of Ukrain; the most important ones are listed below.

Kurochkin et al. [2000] describe the effects of combinations of Ukrain and etoposide in CHO cells.

In Gagliano et al. [2007] the proapoptotic effects of Ukrain in glioblastoma cells are described suggesting some action mechanisms, without however investigating which component(s) of Ukrain is (are) responsible for the desired action and without considering combinations with other drugs.

Funel et al. [2010] disclose an in vitro study that provides information about the action mechanism of Ukrain with respect to cells of ductal pancreatic adenocarcinoma.

The idea of associating Ukrain with other molecules has been described in many papers; see, for example, Skivka et al. [2011], describing in vitro and in vivo studies on an animal model (low- and high-metastasizing melanoma B16 in mice), or Kurochkin et al. [2000], concerning associations of Ukrain with etoposide.

The conclusions of these studies suggest the existence of extreme variability in the effects produced by Ukrain and/or by continuations thereof with other molecules, depending on the type of tumour (and hence of cell line), and this is in accordance with observations carried out also by the present Applicants, which will be further described below.

In Gagliano et al. [2012], the aforesaid study gives important information about the action mechanism of Ukrain with respect to cells of ductal pancreatic adenocarcinoma, without however providing any specific therapeutic indications.

In the art, the effects of extracts of C. majus and of combinations thereof with antitumour drugs have also been investigated.

In Biswas et al [2008] there are described studies on the possible effects of an ethanolic extract of the entire plant as an antitumour, hepatoprotective agent capable of antagonizing the genotoxic effects in case of hepatocarcinogenesis induced in mice by p-dimethylaminoazobenzene (p-DAB). The results show that the extract has the hypothesized properties.

In Nadova et al. [2008] there are described studies aimed at verifying if methanolic extracts of C. majus have antioxidant properties and can inhibit proliferation and induce apoptosis in vitro in leukemia cells. The results confirmed the presence of such properties in the extract.

In Chaadaeva et al. [2009] there is described a study conducted by using extracts of C. majus and of other plants on ASF-LL cells, which constitute a T-lymphoma model in mice. In this regard, it must be pointed out that no reference is made to alkaloids in the article, wherein only peptides contained in the extracts are mentioned.

El-Readi et al [2013] make mention of the effects of extracts of C. majus and its chelidonine component on the phenomenon called “multidrug resistance” (MDR), which is often found in tumour cells and involves several biological targets and various action mechanisms. The cells under investigation were Caco-2 and CEM/ADR5000 (leukemia).

In Capistrano et al. [2015] there is described the effect of crude ethanol extracts or ethanolic extracts deprived of the lipophilic components also soluble in n-hexane. Such extracts were tested in vitro on human and murine tumour cell lines, with results dependent on the cell line type. A normal cell model was also considered, different from the cells used as a non-tumour cell model in the studies described herein.

In experiments carried out in vivo, the second extract seemed to have antimetastatic properties, and chelidonine, sanguinarine, chelerythrine and protopine were found therein. The extract seemed to posses better antimetastatic properties than Ukrain, but it did not reduce the primary tumour. Also this article highlights the extreme variability of the effects of the various compounds or preparations directly obtained from C. majus on the different cell lines used in the tests.

In Park et al. [2015] mention is made of the effects of extracts of C. majus on A431 cells (human epidermoid carcinoma) and the numerous action mechanisms involved.

As stated before, the composition of Ukrain in terms of alkaloids present therein is not completely and satisfactorily known.

Until very recently, no exhaustive studies could be found in the scientific literature that described all of the alkaloids contained in Ukrain, nor the quantities of the most important ones.

According to a number of previous studies, the alkaloids contained in Ukrain are supposed to be about thirty [Táborská et al. 1995], among which chelidonine and coptisine are mentioned as the main ones.

A very recent article [Jesionek et al. 2016], published on line in December 2015, presents an in-depth study on the alkaloids contained in Ukrain, compared with the alkaloids contained in extracts of C. majus; however, not even this very late publication provides an exhaustive quantification or dosage of such alkaloids.

For the sake of completeness, it must be added in this introduction that in the scientific literature concerning studies on the potential anticancer properties of extracts of C. majus, reference is made to extracts that are mostly obtained by using alcoholic solvents such as methanol or ethanol. In some cases, no sufficiently detailed description of the extract preparation procedure is provided. Reference is made in some cases to extracts of roots only, while in some other cases reference is made to extracts of dry plants. In general, substantial variations have been observed in the quantities of the various alkaloids taken into consideration depending on the sample of C. majus (in terms of plant organs employed) and on the solvent in use. The existing literature also mentions variations in the components of the extracts, which are related to the plant picking time. It is therefore apparent that, in the absence of a rigorous standardization in the definitions of the extracts, the literature data concerning the potential antitumour and antimetastatic effects of extracts of C. majus are not comparable with one another, nor can they be compared with the effects of individual alkaloids. For example, it may be useful to mention the differences in the ratios between the quantities of each alkaloid of interest for the work described herein, when the extract is obtained from flowers only, from aerial parts, or from the entire plant (see International patent applications WO 2013/084162 and WO 2013/084163).

The differences between one of these extracts and a mother tincture that can be considered to be sufficiently standardized (Boiron tincture) is shown by way of example in the following table.

TABLE
(Concentrations are expressed in ppm)

| Extract | Berberine | Chelidonine | Protopine |
|---|---|---|---|
| Glycolic Extract | 21.63 | 12.96 | 7.49 |
| Mother Tincture | 0.01 | 29.29 | 0.21 |

Based on the above considerations about the scientific literature concerning Ukrain and extracts of C. majus, no elements can be found which might subtract character of novelty from the work described herein, as will be remarked more in detail below.

In light of the above, it can be stated that are object of the present invention is to provide a combination endowed with antitumour activity and comprising alkaloids of C. majus, which has favourable effects as regards the differential cytotoxicity between pathological cells and healthy cells, so that it can be used to advantage for chemotherapeutic treatments.

This object is achieved by using an alkaloid comprised in the group including berberine, chelidonine and protopine; in particular, it was surprisingly found that the combination of one of these compounds with an antitumour drug such as gemcitabine or the active metabolite of temozolomide (MTIC) increases the efficacy thereof, thereby suggesting the use of dosages that will reduce its toxicity. The reason why reference is made herein to the temozolomide metabolite MTIC instead of temozolomide itself, is that MTIC is the molecule that actually reaches the target at cellular level following administration of temozolomide to a living organism (whether a human being or a test animal). This should not however be understood as a limiting factor that will exclude temozolomide perse or other metabolites thereof.

This effect has been specifically observed in pancreatic cancer cells as regards gemcitabine combined with berberine, and in glial cancer cells as regards the active metabolite of temozolomide (MTIC) combined with alkaloids of C. majus.

This effect was surprising because, in the above-described prior art and from studies conducted in our laboratory, the alkaloids of C. majus, including those considered herein, whether alone or combined together, showed properties that make them suitable for cosmetic applications or anyway for the treatment of skin disorders, since they show either stimulatory activity of skin regeneration or antifibrotic and/or keratolytic activity, other than anti-inflammatory activity [see International patent applications WO 2013/084162 e WO 2013/084163].

Gemcitabine is a well known antitumour drug which is used for, among others, the treatment of pancreatic cancer; it can be used either alone or combined with other compounds (e.g. cisplatin, paclitaxel), depending on the therapy and/or the body organs to be treated (ovaries, breast, etc.), but no therapeutic use thereof is known in association with alkaloids of C. majus.

The same considerations also apply, mutatis mutandis, to temozolomide, which is a well known drug for the treatment of glioblastoma; in particular, all of the above-mentioned alkaloids have shown a surprising reduction in the cell metabolic activity when used in binary combinations with the active metabolite of temozolomide (MTIC).

The features of the invention will be specifically set out in the claims appended to the present description; such features, as well as the effects arising therefore and the advantages provided by the invention, will become more apparent in the light of the following description of a number of exemplary embodiments which are provided by way of non-limiting explanations with reference to the annexed drawings, wherein:

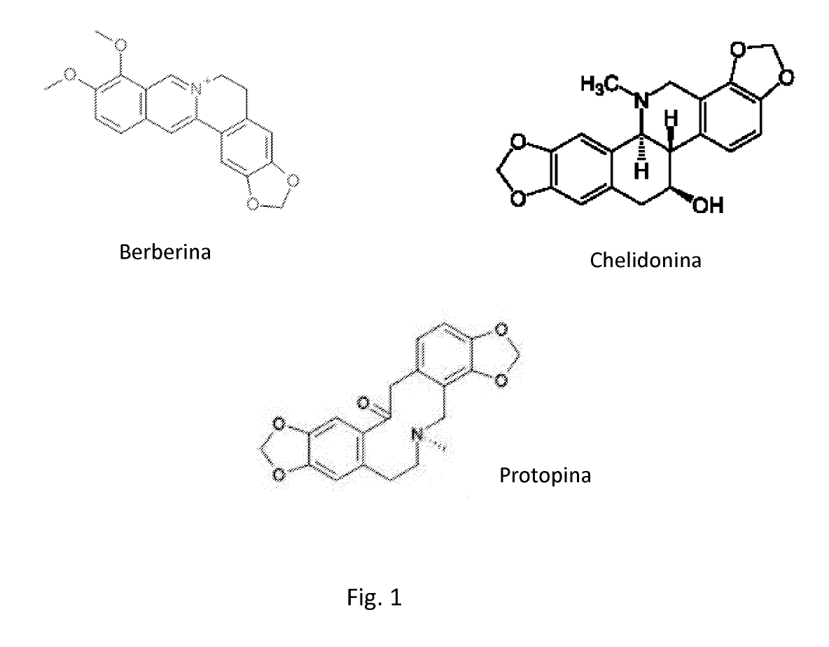

FIG. 1 shows the structural formulae of the alkaloids taken into account for the combination of the invention;

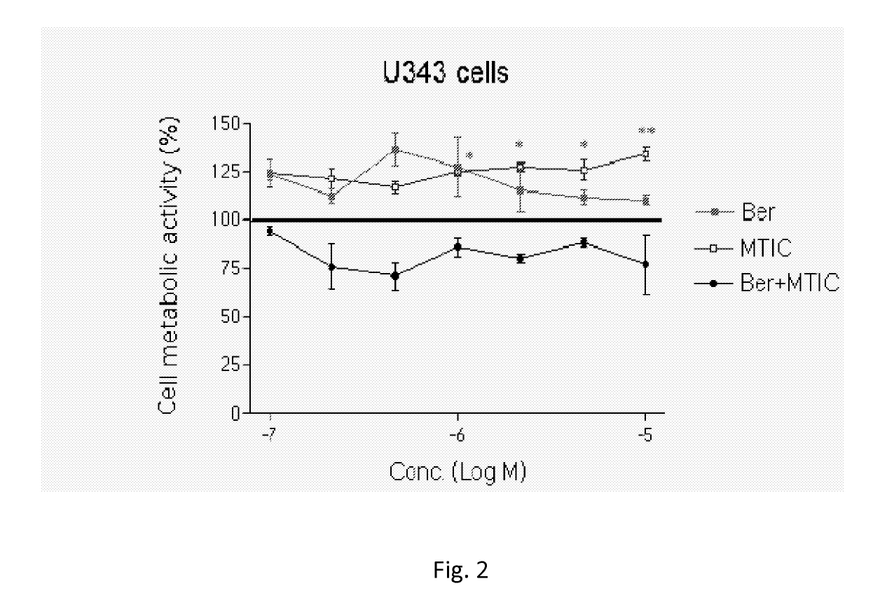

FIG. 2 is a graph that shows the progress of the cell metabolic activity relating to glioblastoma cells with variable concentrations of berberine, of the active metabolite of temozolomide (MTIC), and of combinations thereof;

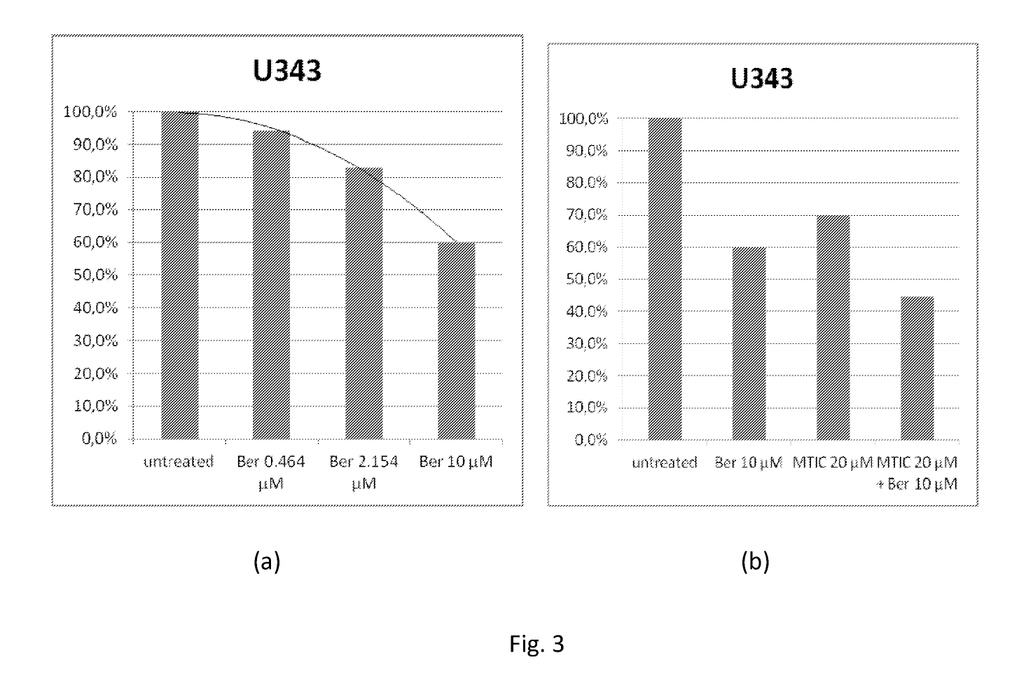

FIG. 3 is a bar diagram that illustrates the viable cell count in assays on glioblastoma cells (U343), wherein: section (a) shows the percentage of viable cells with variable concentrations of individually administered berberine, whereas section (b) shows the percentage of viable cells following administration of active metabolite of temozolomide (MTIC) at a fixed concentration of 20 μM, of berberine at the highest tested concentration (10 μM), and of the combination of MTIC and berberine, wherein berberine is at the highest tested concentration;

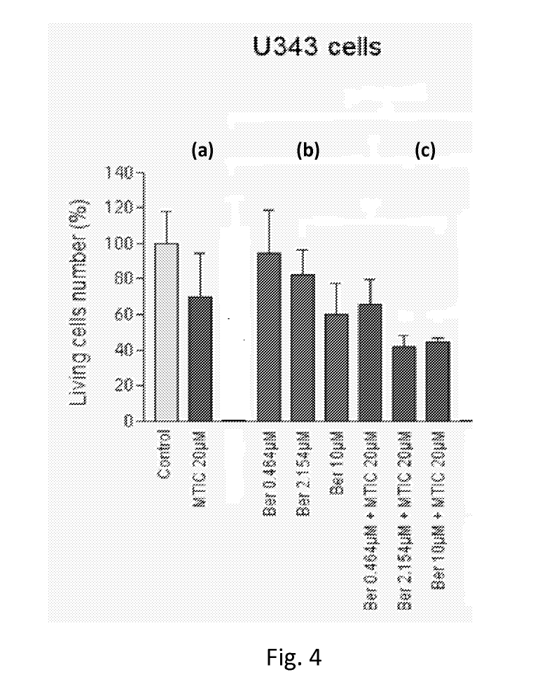

FIG. 4 is a bar diagram that illustrates the percentage of viable cells in assays on glioblastoma cells (U343), wherein: (a) refers to control and treatment with MTIC (at a fixed concentration), (b) refers to variable concentrations of berberine, and (c) refers to treatments with three combinations of active metabolite of temozolomide (MTIC, fixed concentration of 20 Mμ) and berberine at the three different concentrations taken into account;

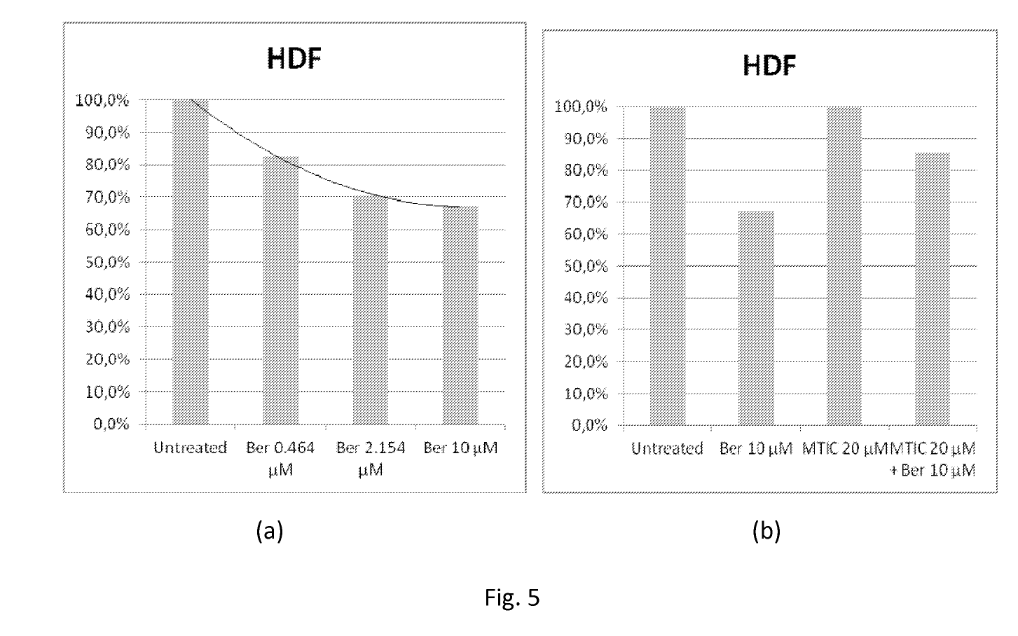

FIG. 5 is a bar diagram that shows the percentage of viable cells in assays on human dermal fibroblasts (HDF), (a) when there are treated with a changing concentration of berberine administered individually whereas (b) illustrates the percentage of viable cells following administration of berberine at the highest concentration tested (10 microM), the temozolomide active metabolite (MTIC) used at a fixed concentration (equal to 20 microM), and the combination of MTIC with berberine, in which berberine is present at the highest concentration tested;

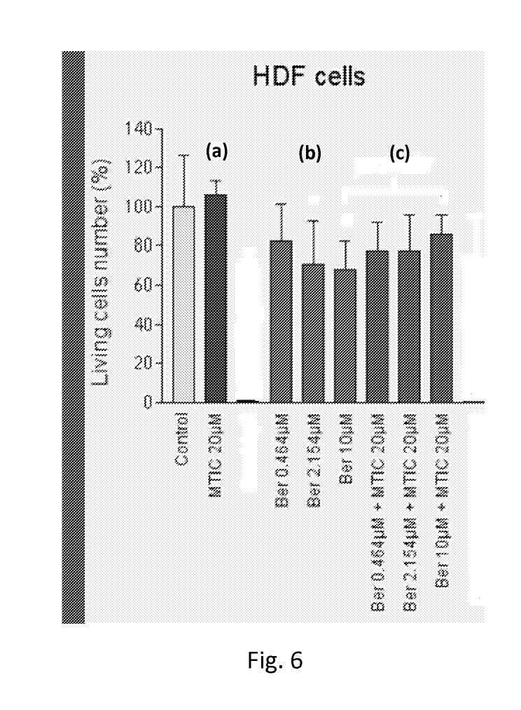

FIG. 6 is a bar diagram that shows the percentage of viable cells in assays on human dermal fibroblasts (HDF), when treated (a) with a fixed concentration of MTIC, (b) with berberine at three different concentrations, and (c) with combinations of MTIC and berberine, wherein the latter is used at the three different concentrations at which it was individually tested;

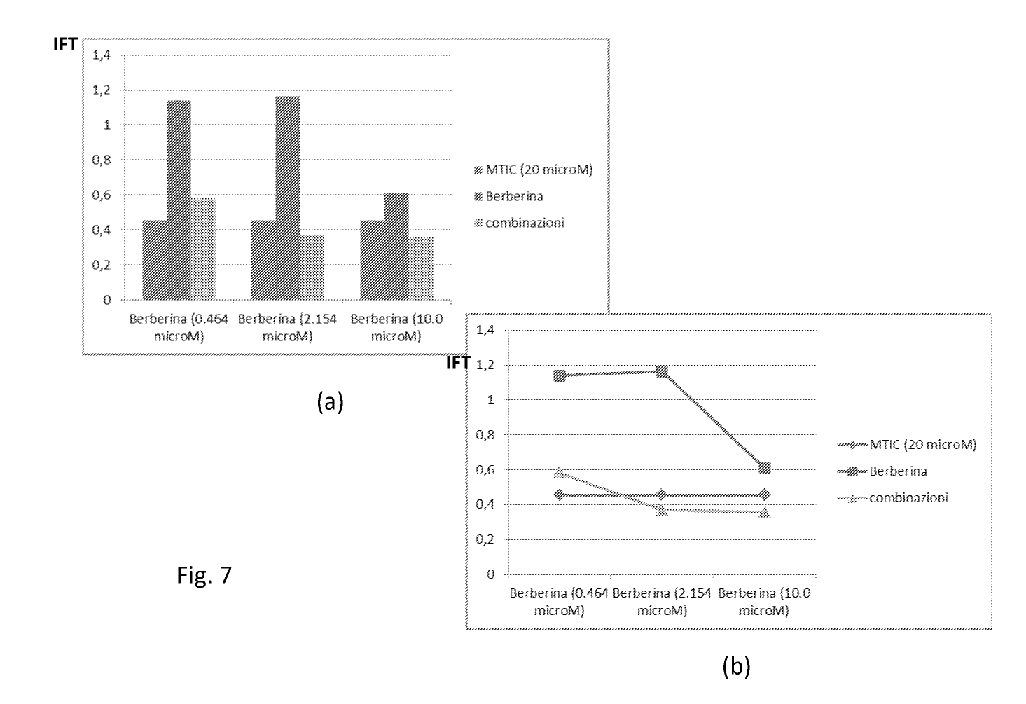

FIG. 7 contains two graphs that schematize the tread of a parameter (“therapeutic favourability index”—TFI, defined in detail in EXAMPLE 2), consisting of the ratio between the percentage of tumour cells and the percentage of cells chosen as a non-tumour cell model that remain viable following the various assayed treatments;

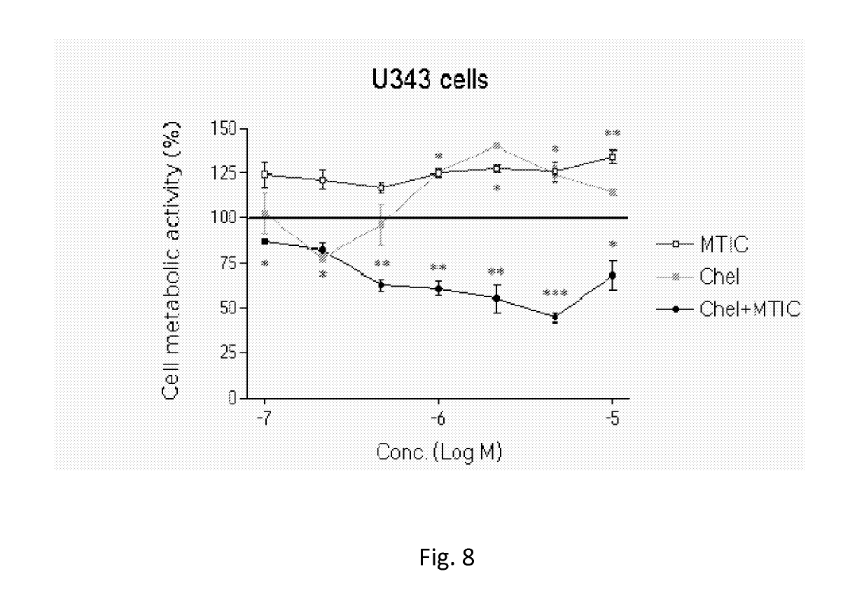

FIG. 8 is a graph that shows the progress of the metabolic activity relating to glioblastoma cells with variable concentrations of chelidonine, of the active metabolite of temozolomide (MTIC), and of the combination thereof;

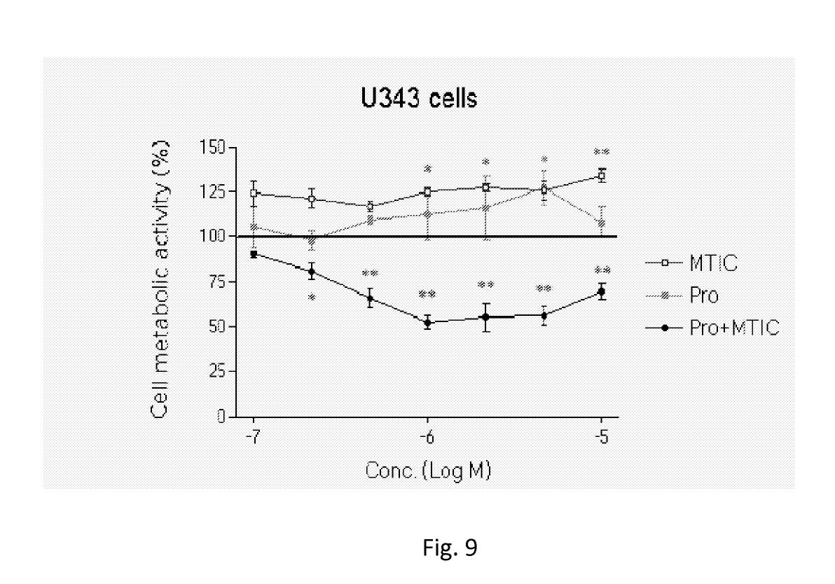

FIG. 9 is a graph that shows the progress of the metabolic activity relating to glioblastoma cells with variable concentrations of protopine, of the active metabolite of temozolomide (MTIC), and of the combination thereof;

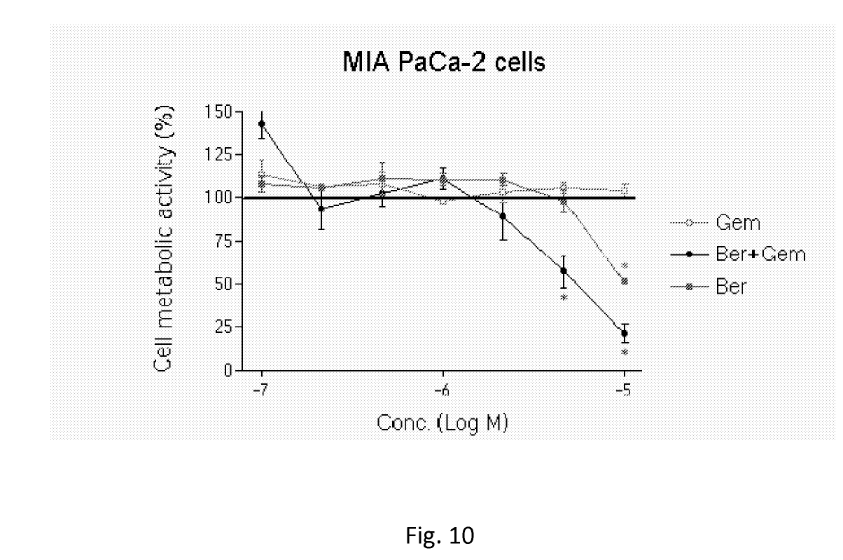

FIG. 10 is a graph that shows the progress of the metabolic activity relating to pancreatic tumour cells (MIA PaCa-2) with variable concentrations of berberine, of gemcitabine, and of the combination thereof;

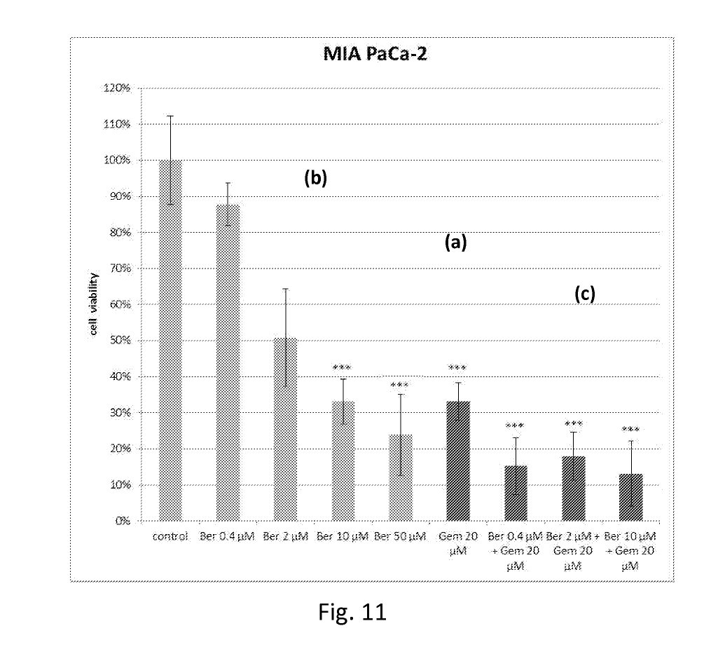

FIG. 11 is a graph that shows the percentage of pancreatic tumour cells (MIA PaCa-2) that remain viable after the following treatments: (a) with gemcitabine alone (used at a fixed concentration of 20 μM), (b) with variable concentrations of berberine (0.4 μM, 2.0 μM, 10.0 μM and 50.0 μM), and (c) following treatments with three combinations containing gemcitabine (at a fixed concentration) and berberine at concentrations of 0.4 μM, 2.0 μM, 10.0 μM;

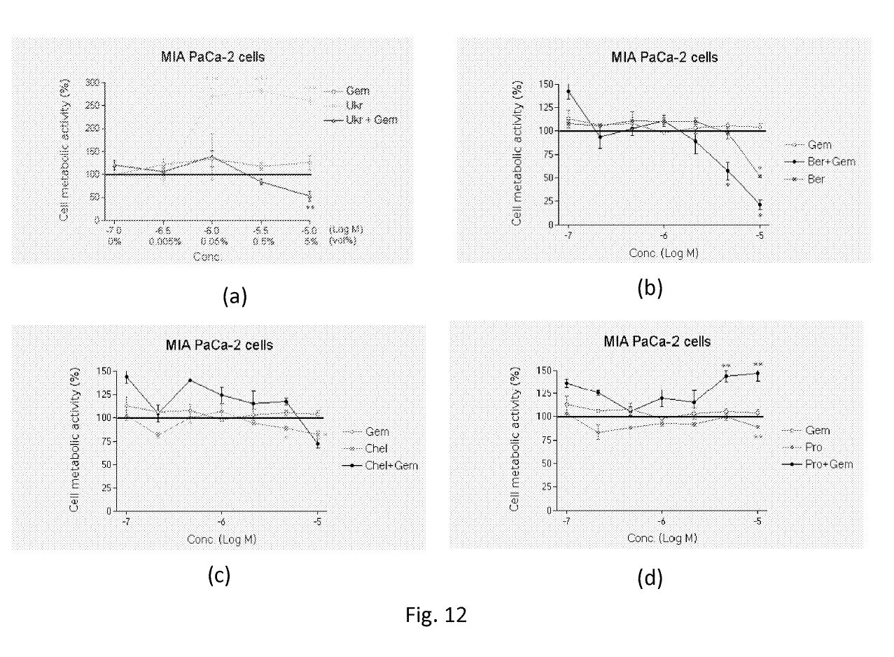

FIG. 12 is a graph that shows the progress of the cell metabolic activity relating to MIA PaCa 2 cells after the following treatments: (a) with Ukrain alone, with gemcitabine alone, and with an association between Ukrain and gemcitabine, (b) with gemcitabine alone, with berberine alone, and with an association between gemcitabine and berberine, (c) with gemcitabine alone, with chelidonine alone, and with an association between gemcitabine and chelidonine, and (d) with gemcitabine alone, with protopine alone, and with an association between gemcitabine and protopine at variable concentrations.

With reference to the drawings, the alkaloids taken into consideration in the first figure are of the type commercially available (in this case, from Sigma-Aldrich) and have the highlighted structure.

The same applies to the antitumour drugs that were assayed in combination with the alkaloids, i.e. gemcitabine (supplied by Sigma-Aldrich) and the active metabolite of temozolomide MTIC [5-(3-methyl-1-triazeno)imidazole-4-carboxamide], supplied by Santa Cruz Tech.

In order to evaluate the effects of the antitumour combination of the invention, cellular viability was taken into account as a switable reference parameter, observed after in vitro application of the antitumour combination and estimated in terms of dehydrogenase activity. Moreover, in the case of treatments on U343 cells with berberine and associations thereof with MTIC, and in the case of treatments on MIA PaCa-2 cells with berberine and associations thereof with gemcitabine, also the percentage of viable cells, estimated via cell count, was taken into consideration.

For sake of completeness' of information and for the purpose of evaluating the differential cytotoxicity exerted both by the individual components of the combinations and by the combinations themselves on tumour and normal cells, the same treatments tested on U343 cells were also conducted on human dermal fibroblasts (HDF), chosen as a model of cells not concerned by tumour pathologies; this allowed, in the case of treatments tested on U343 cells, evaluations of the differential effects of the tested agents against tumour cells and against a model of normal cells, in accordance with the invention.

Furthermore, cell viability was estimated in two different ways: dehydrogenase assay, based on the reaction of formazan (WST-1), and viable cell count with Neubauer chamber after treatment with Trypan blue.

The results obtained showed a sensible reduction in the viability of tumour cells when the alkaloids are used in combination with antitumour drugs; such a reduction is surprisingly present in the case of application to glioblastoma cells, also for low concentrations of the compound combination.

In the case of pancreatic tumour cells, a reduction in their viability was observed with increased concentrations of the combination of gemcitabine and the alkaloid berberine.

A comparison with the application of the same antitumour combinations on non-tumour cells, such as human dermal fibroblasts (HDF), surprisingly showed that the combination, which per se proves to be more effective than the individual components on tumour cells, is also significantly less toxic on the HDF cells chosen as a normal cell model.

What represents an important aspect of the invention is the discovery of the differential reduction effect on cell viability, based on which it can be stated that the cytotoxicity produced on U343 cells by the association of MTIC with berberine is significantly more marked than the cytotoxicity produced by the same association on the normal HDF cell model, compared to the effects produced by the single components taken individually (MTIC and berberine). This makes the combination suitable for therapeutic uses.

During the work conducted for this patent application, the presence in Ukrain of the 7 alkaloids contained in the extracts of C. majus mentioned in International patent applications WO2013084162 and WO2013084163 was investigated for sake of scrupulousses, to quantify them in Ukrain if they were present. For sake of completeness, the following table shows the results of our quantitative study on the alkaloids of C. majus which are of interest for the study described herein.

| | Berberine | Chelidonine | Protopine |
|---|---|---|---|
| | Molar Conc. | Molar Conc. | Molar Conc. |
| Ukrain | 4.69 × 10⁻⁶ | 7.71 × 10⁻⁵ | 3.24 × 10⁻⁴ |

As a confirmation of the extreme variability of the responses evoked by a same molecule or by a same combination of individual components on different cell lines, even of the same type of tumour pathology, which emerges from an analysis of the scientific literature, we have deemed it useful to conduct specific experiments, the results of which are shown in FIG. 12 and commented below.

For example, the association with gemcitabine of each one of the alkaloids mentioned in this application represents a novelty, in that the experiments have shown that the effects of the association of Ukrain with gemcitabine, on one of the two investigated cell lines (MIA PaCa-2—pancreatic cancer), are very different from the effects produced by binary associations of gemcitabine with each one of the three alkaloids taken into account (see graphs shown in FIG. 12), and this finding means that the behaviour of the associations was not foreseeable on the basis of prior knowledge [e.g. from Gansauge et al. 2002, Chinese patent application CN 103 372 210, or from Fan Li-Xia Biochimica et Biophysica Acta (BBA) 2013, Ernst et al. 2005].

Indeed, the graph (a) in FIG. 12 shows that, when Ukrain (yellow line) is administered individually to MIA PaCa-2 cells at percent concentrations higher than 0.005% (v/v), cell viability increases (this effect is opposite to the desired one), and then it remains relatively constant starting from a percent concentration of 0.05% until the highest tested concentration (5%) is reached. The concentration of Ukrain is expressed as a percent volume relative to the sample in solution directly supplied by the producer.

The binary association of Ukrain with gemcitabine begins to cause a reduction in the viability of MIA PaCa-2 cells (desired effect) starting from a concentration slightly lower than 10⁻⁵⁵ M of gemcitabine and slightly lower than 0.5% of Ukrain.

Binary associations of gemcitabine with berberine [FIG. 12 (b)] and of gemcitabine with chelidonine [FIG. 12 (c)] show a similar reduction in the viability of MIA PaCa-2 cells (desired effect) in the area of the highest concentrations considered in the graph, while the binary association of gemcitabine with protopine [FIG. 12 (d)] seems to increase cell viability (undesired effect) in an irregular manner in most of the assayed concentrations, without ever causing it to diminish.

As regards the effects produced on MIA PaCa-2 cells by combinations of individual alkaloids of C. majus with gemcitabine, it can be concluded that, while binary associations of gemcitabine with berberine and with chelidonine produce, at certain concentrations only and with different profiles, similar effects consisting of a reduction in the viability of the MIA PaCa-2 cells compared to the treatment with gemcitabine alone or with the individual alkaloid, protopine associated with the same molecule (gemcitabine) actually causes an increase in the viability of the same cells.

The substantial and unpredictable difference that it was been observed between the effects produced by Ukrain and the effects produced by individual alkaloids, is also supported by the differential results observed and described in a previously mentioned article [Habermehl et al. 2006] concerning to the effects produced on Jurkat T-lymphoma cells by the alkaloids under examination within that context (including chelidonine and protopine, but not berberine).

### EXAMPLE 1 (EXPERIMENTS BASED ON WST-1 ASSAY)

Alkaloids of C. majus (berberine, chelidonine, protopine), gemcitabine, Trypsin-EDTA enzyme, fetal bovine serum (FBS), dimethyl sulfoxide (DMSO), L-Glutamine and Trypan blue were purchased from Sigma-Aldrich.

Metabolite (5-(3-methyl-1-trizeno)imidazole-4-carboxamide) of temozolomide MTIC was supplied by Santa Cruz Tech.

MIA PaCa-2 pancreatic tumour cell lines and glioblastoma cell lines (U343) were initially obtained from the Pharmacy Department of the University of Pisa and subsequently purchased.

Cell Proliferation Reagent (WST-1) was purchased from Roche Diagnostics GmbH (Manneheim, Germany), whereas human dermal fibroblast cells (HDF) came from ATCC.

All agents were dissolved into DMSO and preserved at −20° C. for a short time prior to use. The cells were cultivated in monolayer culture in DMEM ground (Life Technologies), integrated with 5% fetal bovine serum (FBS), 1% L-glutamine and 1% antibiotics (penicillin-streptomycin from Lonza) at 37° C. in humidified 5% CO₂ atmosphere.

The MIA PaCa-2 pancreatic tumour cells and the U343 glioblastoma cells were cultured in 96-well plates for cell culture until an approximate 70-80% confluence, after 4 hours and treated with the agents. The variation of cell viability was estimated after 48 hours via WST assay, wherein WST-1 was used as a reagent. The absorbance reading was taken at 450 nm. Measurements were taken for different agent concentrations on:

(a) untreated cells 0 hours after adhesion (control at time zero, controlₜ₀),

(b) untreated cells after a 48-hour interval from adhesion (control after 48 hours, controlₜ₄₈),

(c) cells treated with the agents of interest and observed 48-hour after adhesion.

The cell viability variation was set to 0% for control cells at t=0; it was set to 100% for control cells at t=48 hours. All agent concentrations were assayed in triplicate.

The following tables show the data from which the illustrated graphs were derived.

In particular, Table 1 shows the data relating to berberine, MTIC and the combination thereof, applied to the U343 glioblastoma cell line and obtained via WST assay, from which the graph of FIG. 2 was derived. The latter indicates on the X axis the concentration of the assayed agents. As far as the binary association is concerned, its components have a fixed 50/50 ratio.

TABLE 1

| Type of Experiment: | WST assay | | |
|---|---|---|---|
| Cell line: | U343 (glioblastoma) | | |
| Compounds: | Berberine | MTIC | MTIC + Berberine |
| Concentrations: | 0.1 μM-μ10M | 0.1 μM-10 μM | Equimolar |

| | Berberine | | | MTIC | | | Ber + MTIC | | |
|---|---|---|---|---|---|---|---|---|---|
| | Δ | | | Δ | | | Δ | | |
| | (viability %) | SEM | n | (viability %) | SEM | n | (viability %) | SEM | n |
| 10 μM | 10.3533 | 2.79164 | 3 | 34.009 | 3.4029 | 3 | −23.25687 | 15.5957 | 3 |
| 4.642 μM | 11.3394 | 3.87418 | 3 | 25.991 | 5.2831 | 3 | −11.56095 | 2.16065 | 3 |
| 2.154 μM | 15.4889 | 11.3021 | 3 | 27.2523 | 2.6138 | 3 | −19.83807 | 2.45443 | 3 |
| 1 μM | 27.3008 | 15.3451 | 2 | 24.9099 | 2.4577 | 3 | −14.30499 | 4.78676 | 3 |
| 0.464 μM | 36.3599 | 8.01151 | 2 | 16.8018 | 3.0341 | 3 | −29.32973 | 7.17763 | 3 |
| 0.215 μM | 12.3254 | 3.98817 | 3 | 21.1712 | 5.0166 | 3 | −24.2915 | 11.4761 | 3 |
| 0.1 μM | 23.2128 | 2.8618 | 3 | 24.009 | 7.1852 | 3 | −5.48808 | 2.29948 | 3 |

From Table 1 it emerges that, in U343 cells, administration of MTIC increases the cell metabolic activity in an irregular manner (from approx. 115 up to 130%, depending on MTIC concentration), and that administration of berberine alone causes an increase, though not a very marked one, in the metabolic activity (from approx. 110 to 136%). Equimolar combinations of MTIC and berberine show, on the contrary, a reduction in the cell metabolic activity (between 90 and 70%), which at certain concentrations appears to be relevant.

Table 2 shows the data relating to, respectively, chelidonine, MTIC and the combination thereof, applied to the U343 glioblastoma cell line and obtained via WST assay, from which the graph of FIG. 8 was derived; the variables shown on the X and Y axes of the latter are the same as those in the graph of FIG. 2.

TABLE 2

| Type of Experiment: | WST assay | | |
|---|---|---|---|
| Cell line: | U343 (glioblastoma) | | |
| Compounds: | Chelidonine | MTIC | MTIC + Chelidonine |
| Concentrations: | 0.1 μM-10 μM | 0.1 μM-10 μM | Equimolar |

| | Chelidonine | | | MTIC | | | Chel + MTIC | | |
|---|---|---|---|---|---|---|---|---|---|
| | Δ | | | Δ | | | Δ | | |
| | (viability %) | SEM | n | (viability %) | SEM | n | (viability %) | SEM | n |
| 10 μM | 14.3799 | 1.97947 | 3 | 34.009 | 3.4029 | 3 | −31.94325 | 8.51648 | 3 |
| 4.642 μM | 24.4335 | 4.67778 | 3 | 25.991 | 5.2831 | 3 | −55.546 | 2.35753 | 3 |
| 2.154 μM | 40.0495 | 0 | 1 | 27.2523 | 2.6138 | 3 | −44.96991 | 7.95217 | 3 |
| 1 μM | 25.9168 | 0.47518 | 3 | 24.9099 | 2.4577 | 3 | −39.29492 | 3.96159 | 3 |
| 0.464 μM | −3.74948 | 11.7124 | 3 | 16.8018 | 3.0341 | 3 | −37.44626 | 3.3534 | 2 |
| 0.215 μM | −22.4145 | 2.15087 | 3 | 21.1712 | 5.0166 | 3 | −17.92777 | 3.61314 | 3 |
| 0.1 μM | 2.4722 | 11.2485 | 2 | 24.009 | 7.1852 | 3 | −13.3706 | 1.53393 | 3 |

From Table 2 it emerges that, in the same U343 cells, administration of chelidonine alone produces effects characterized by an irregular trend, with a reduction, at specific concentrations only, in the metabolic activity. Equimolar binary combinations of MTIC and chelidonine cause a significant decrease in the metabolic activity (between 85 and 45%) in a wide range of tested concentrations.

Table 3 shows the data relating to, respectively, protopine, MTIC and the combination thereof, applied to the U343 glioblastoma cell line and obtained via WST assay, from which the graph of FIG. 9 was derived; the variables shown on the X and Y axes are the same as those in the graph of FIG. 2.

TABLE 3

| Type of Experiment: | WST assay | | |
|---|---|---|---|
| Cell line: | U343 (glioblastoma) | | |
| Compounds: | Protopine | MTIC | MTIC + Protopine |
| Concentrations: | 0.1 μM-10 μM | 0.1 μM-10 μM | Equimolar |

| | Protopine | | | MTIC | | | Pro + MTIC | | |
|---|---|---|---|---|---|---|---|---|---|
| | Δ | | | Δ | | | Δ | | |
| | (viability %) | SEM | n | (viability %) | SEM | n | (viability %) | SEM | n |
| 10 μM | 7.968 | 8.62211 | 3 | 34.009 | 3.4029 | 3 | −30.73356 | 4.50149 | 3 |
| 4.642 μM | 27.3609 | 9.83487 | 3 | 25.991 | 5.2831 | 3 | −44.09781 | 4.99556 | 3 |
| 2.154 μM | 16.231 | 18.0266 | 3 | 27.2523 | 2.6138 | 3 | −44.98314 | 7.70717 | 3 |
| 1 μM | 12.0152 | 14.2498 | 3 | 24.9099 | 2.4577 | 3 | −47.84991 | 3.96806 | 3 |
| 0.464 μM | 9.1484 | 2.11928 | 3 | 16.8018 | 3.0341 | 3 | −34.19056 | 5.36789 | 3 |
| 0.215 μM | −2.02362 | 5.50826 | 3 | 21.1712 | 5.0166 | 3 | −19.13996 | 4.56248 | 3 |
| 0.1 μM | 5.8179 | 11.2637 | 3 | 24.009 | 7.1852 | 3 | −9.73862 | 2.04501 | 3 |

From Table 3 it emerges that, in the same U343 cells, administration of protopine alone causes a slight increase in the metabolic activity (up to 130%), whereas the equimolar binary combination of MTIC and protopine causes a decrease in the metabolic activity (between 80 ad 50%) at all of the assayed concentrations, which turns out to be particularly marked at concentrations in the range of 10^{-6.5} M to 10^{-5.5} M.

Table 4 shows the data of berberine, gemcitabine and the combination thereof, applied to the MIA PaCa-2 pancreatic tumour line and obtained via WST assay, from which the graph of FIG. 10 has been drawn.

TABLE 4

| Type of Experiment: | WST assay | | |
|---|---|---|---|
| Cell line: | MIA PaCa-2 (pancreatic carcinoma) | | |
| Samples: | Berberine | Gemcitabine | Gem + Berberine |
| Concentrations: | 0.1 μM-10 μM | 0.1 μM-10 μM | Equimolar |

| | Berberine | | | Gemcitabine | | | Ber + Gem | | |
|---|---|---|---|---|---|---|---|---|---|
| | Δ | | | Δ | | | Δ | | |
| | (viability %) | SEM | n | (viability %) | SEM | n | (viability %) | SEM | n |
| 10 μM | −64.76 | 8.38 | 2 | −2.25 | 4.4 | 3 | −84.26 | 3.28 | 3 |
| 4.642 μM | −69.29 | 5.96 | 2 | 24.16 | 11.47 | 3 | −57.97 | 3.24 | 2 |
| 2.154 μM | -3.42 | 7.85 | 2 | 17.64 | 7.84 | 3 | −6.44 | 2.72 | 2 |
| 1 μM | −16 | 3.79 | 3 | 3.26 | 2.7 | 3 | 1.18 | 8.53 | 2 |
| 0.464 μM | −10.41 | 7.66 | 3 | 12.56 | 21.83 | 2 | −12.22 | 6.94 | 3 |
| 0.215 μM | 5.73 | 3.05 | 3 | 27.87 | 11.67 | 3 | −13.33 | 1.93 | 3 |
| 0.1 μM | −11.32 | 3.96 | 3 | 54.72 | 7.33 | 3 | −5.08 | 2.35 | 3 |

From Table 4 it emerges that, in MIA PaCa-2 cells, wherein the treatment with gemcitabine alone causes an essentially negligible increase in the cell metabolic activity, individually administered berberine causes a significant decrease in the metabolic activity only starting from concentrations slightly lower than 10 μM (−5 Log M) (approx. 50%). Equimolar combinations of gemcitabine and berberine produce a considerable reduction in the metabolic activity in a dose-dependent manner, starting from individual component concentrations of approx. 4.64 μM (−5.33 Log M) and going towards higher concentrations. The treatment with the combination wherein the individual components' concentration is 4.64 μM reduces the metabolic activity to 57%, while the treatment with the combination wherein the individual components' concentration is 10 μM reduces the metabolic activity to 21%.

It must be pointed out that, as far as the diagnostic value of the WST assay is concerned, instead of generally speaking of cell viability it would be appropriate to refer to cell metabolic activity monitored via cellular dehydrogenase activity.

### EXAMPLE 2 (EXPERIMENTS BASED ON U343 CELL COUNT)

The U343 cells and the HDF cells were cultivated in T25 flasks and treated with the agents after reaching a confluence of 90%. After 48 hours of treatment, the cells were detached with trypsin-EDTA and suspended in medium consisting of 50% DMEM and 50% Trypan blue. The number of viable cells after each treatment was estimated by using the Neubauer counting chamber, excluding cells positive to Trypan blue. At least three countings were made for each treatment.

The percentage of viable cells compared to the control was determined by the ratio between the number of treated cells and the number of untreated cells.

The following tables show the data from which the graphs of FIGS. 3, 4, 5, 6 and 7 were derived.

In particular, the following Table 5 shows the data relating to berberine, MTIC and three combinations thereof (wherein the MTIC concentration is kept fixed, while the berberine concentration varies), applied to the U343 glioblastoma cell line. The data were obtained via cell counting assay, from which the bar diagrams of FIGS. 3 and 4 were derived.

TABLE 5

| Type of Experiment: | Cell counting | | |
|---|---|---|---|
| Cell line: | U343 (glioblastoma) | | |
| Compounds: | Compound I | MTIC | MTIC + Compound I |
| Concentrations: | 0.464 μM-10 μM | 20 μM | 0.464 μM-10 μM/20 μM |

The Figure summarizes the results of the “cell counting” experiment relating to
U343 cells subjected to berberine treatments for 48 hours.

| | Ber | Ber | Ber | MTIC | MTIC + | MTIC + | MTIC + |
|---|---|---|---|---|---|---|---|
| Untreated | 0.464 μM | 2.154 μM | 10 μM | 20 μM | Ber 0.4 μM | Ber 2 μM | Ber 10 μM |
| 100% | 94% | 82.6% | 59.8% | 69.7% | 65.6% | 41.5% | 44.8% |

Table 6 below shows the data relating to berberine, MTIC and three combinations thereof (wherein the MTIC concentration is kept fixed, while the berberine concentration varies), applied to the HDF cell line (normal non-tumour cell model). The data were obtained via cell counting assay, from which the bar diagrams of FIGS. 5 and 6 were derived.

TABLE 6

| Type of Experiment: | Cell counting | | |
|---|---|---|---|
| Cell line: | HDF | | |
| Compounds: | Compound I | MTIC | MTIC + Compound I |
| Concentrations: | 0.464 μM-10 μM | 20 μM | 0.464 μM-10 μM/20 μM |

The Figure summarizes the results of the “cell counting” experiment relating to
HDF cells subjected to berberine treatments for 48 hours.

| | Ber | Ber | Ber | MTIC | MTIC + | MTIC + | MTIC + |
|---|---|---|---|---|---|---|---|
| Untreated | 0.464 μM | 2.154 μM | 10 μM | 20 μM | Ber 0.4 μM | Ber 2 μM | Ber 10 μM |
| 100% | 82.65 | 70.41 | 67.35 | 106.12 | 77.55 | 77.55 | 85.71 |

The graph of FIG. 3, which concerns the treatment of U343 cells, is divided into two parts: the part on the left (a), relating to berberine only, shows on the X axis the berberine concentration and on the Y axis the percentage of viable cells, whereas the part on the right (b) shows the percentage of viable cells after a treatment with the berberine-MTIC combination, wherein berberine has a concentration of 10 μM, at which concentration berberine alone causes the presence of a lower percentage of viable cells. The same graph also shows, for comparison, the percentages of viable cells detected after treatments with 10 μM berberine alone and with 20 μM MTIC alone.

The graph of FIG. 4 shows the effects of treatments carried out on U343 cells, indicating, in addition to the control, the percentage of viable cells after treatments with (a) MTIC administered individually at a concentration of 20 μM, (b) berberine administered individually at three different concentrations (0.464 μM, 2.154 μM and 10 μM), and (c) three binary combinations of 20 μM MTIC with berberine at the above-mentioned three different concentrations (Combination I, containing 0.464 μM berberine—Combination II, containing 2.15 μM berberine—Combination III, containing 10.0 μM berberine).

The graph of FIG. 5 shows the percentage of viable cells in experiments carried out on HDF cells, chosen as a normal cell model, after the same treatments already described for the U343 cells (and partly summarized in the graph of FIG. 3b).

The graph of FIG. 6 shows the effects of treatments carried out on HDF cells chosen as a normal (non-tumour) cell model, indicating, in addition to the control, the percentage of viable cells after treatments with (a) MTIC administered individually at a concentration of 20 μM, (b) berberine administered individually at three different concentrations (0.464 μM, 2.154 μM and 10 μM), and (c) three binary combinations of 20 μM MTIC with berberine at the above-mentioned three different concentrations (Combination I, containing 0.464 μM berberine—Combination II, containing 2.15 μM berberine—Combination III, containing 10.0 μM berberine).

It is important to highlight the fact that cell counting experiments are especially useful to allow estimating the percentage of viable cells (U343 glioblastoma cells and HDF cells, chosen as a normal cell model) that can be observed after the treatments, and that the comparison between the effects on tumour cell lines and those on normal cells allows evaluating, at the same time, the cytotoxicity on the tumour cell line (desired effect) and the cytotoxicity on the normal cell model (undesired effect).

For the purpose of properly interpreting the results of the experiments as a whole, it was therefore useful to define an index that was called “Therapeutic Favourability Index” (TFI), consisting of the ratio between the percentage of viable cells, measured against the control, found after the treatment with the samples under examination in the U343 cells (the proliferation of which must be inhibited) and in the HDF cells (the proliferation of which should ideally remain unchanged compared to the respective control).

Table 7 shows the percentages of viable cells relative to the control that were observed in experiments conducted with U343 cells and HDF cells after administration of MTIC alone (the active metabolite of Temozolomide) (at a concentration of 20 μM), berberine alone (at concentrations of 0.464 μM, 2.15 μM and 10.0 μM, respectively), and combinations consisting of MTIC (20 μM) and berberine (0.464 μM of berberine in combination I, 2.15 μM of berberine in combination II, and 10.0 μM of berberine in combination III, respectively). For each treatment, the therapeutic favourability index (TFI) is also shown, as defined above.

TABLE 7

| | | | TFI index |
|---|---|---|---|
| | % proliferation | % proliferation | U343/HDF |
| | of U343 | of HDF | % proliferation |
| Assayed agent | against control | against control | ratio |
| MTIC (20 μM) | 69.70 | 106.12 | 0.657 |
| Berberine (0.464 μM) | 94.00 | 82.65 | 1.137 |
| Berberine (2.154 μM) | 82.60 | 71.41 | 1.173 |
| Berberine (10.0 μM) | 59.80 | 67.35 | 0.888 |
| Combination I | 65.60 | 77.55 | 0.846 |
| MTIC (20 μM) + | | | |
| Berberine (0.464 μM) | | | |
| Combination II | 41.50 | 77.55 | 0.535 |
| MTIC (20 μM) + | | | |
| Berberine (2.154 μM) | | | |
| Combination III | 44.80 | 85.71 | 0.523 |
| MTIC (20 μM) + | | | |
| Berberine (10.0 μM) | | | |

FIG. 7 shows two graphs that schematize the trend of the TFI parameter for each one of the agents used in the assays. In bar graph (a), the light blue bars refer to MTIC (the concentration of which was set to 20 μM for both individual administration and combinations with berberine), the magenta bars refer to berberine administered individually (and assayed at three different concentrations, i.e. 0.464 μM, 2.154 μM and 10 μM), and the green bars refer to the three combinations wherein the MTIC concentration was kept constant and the berberine concentrations varied in accordance with those used for individual administration of the same. The same values are shown in the dot graph (b). It may be useful to remind that the TFI parameter allows estimating the ratio between desired cytotoxicity (on U343 cells) and undesired cytotoxicity (on HDF cells used as a normal cell model). The lower the value of the TFI parameter, the more the assayed agent is adequate for therapeutic use, since this parameter measures the ratio between desired cytotoxicity (on tumour cells) and undesired cytotoxicity (on the cells of the non-tumour model).

According to the values of the TFI parameter, it turns out that the combination II, and especially the combination III, are expected to produce a significantly better therapeutic effect than administration of temozolomide alone, which reaches the cell in the organism in the form of its MTIC metabolite, and administration of berberine alone. It can also be observed that, on the pair consisting of the U343 cell line (glioblastoma cells) and the HDF cell line (normal cell model), berberine alone would have a worse TFI value than the active metabolite of temozolomide (MTIC). This contributes to giving a character of novelty to the work described herein, compared to the contents of [Liu et al. 2015], published on line on Dec. 12, 2014.

The data reported in [Liu et al. 2015] do not appear to be directly comparable with the results described herein. In such article, in fact, reference is made to the fact that berberine can markedly stop the proliferation of glioblastoma cells more efficiently than temozolomide, since it induces senescence in human glioma cells. More specifically, for the work described in such article the U87, U251 and U118 glioblastoma cell lines were used in addition to the SGH.44 line, and the agents were assayed individually only, at concentrations higher than those considered in the present work.

More in detail, in such article the berberine concentrations used were in the range of 15 to 150 μM. Instead, in the study presented herein, wherein the effects of the agents of interest on the U343 glioblastoma cell line were observed, the maximum berberine concentration used for both individual administration and combinations with 20 μM MTIC did not exceed the value of 10 μM.

Furthermore, in [Liu et al. 2015] the effects of berberine and those of temozolomide were compared, also in the in vitro experiments (experiments on cells), by directly using temozolomide (prodrug), without taking into account the fact that, after administration to a living organism, the molecule that reaches the cell to exert its effect is actually its active metabolite MTIC. Therefore, the use of temozolomide is correct for in vivo experiments, but, based on the above consideration, the choice of the temozolomide prodrug does not appear to be appropriate for in vitro experiments. In addition, in [Liu et al. 2015] the temozolomide directly administered to the cells was used in the concentration range of 40 to 320 μM. In the study presented herein, instead, the MTIC concentration used for both individual administration and combinations with berberine at the three different concentrations specified for the Combinations I, II and III was 20 μM.

In the study described herein, temozolomide was also assayed by direct administration to U343 cells and HDF cells, for the purpose of observing the direct effect of temozolomide on such cells. It was possible to observe that direct administration of temozolomide does not cause significant variations (within the measurement accuracy limits) in the percentage of viable U343 cells, while it causes a decrease up to a 60-70% reduction in the percentage of viable HDF cells.

In the study presented herein, in order to compare the effects exerted on the cells by the administration of temozolomide, it was deemed to be more appropriate to use the active metabolite of temozolomide (MTIC), since this is the molecule that actually reaches the cell after administration of temozolomide to a living organism (whether a human being or a test animal).

As aforementioned, in [Liu et al. 2015] the concentrations used for the agents under investigation are higher than those used in the study described herein, and, since one of the objects of this study was to identify a combination of agents that would prove effective for the treatment of glioblastoma while reducing the inevitable side effects due to administration of temozolomide, the contribution of this study to finding a therapy that, as a whole, is more favourable for the patients takes a character of novelty. This is true not only because it suggests the use of combinations as opposed to berberine alone, but also because it suggests the use of reduced drug dosages.

It is also important to point out that it cannot be automatically predicted that the combination of berberine and MTIC (in in vitro experiments) or the administration (in vivo) of combinations of berberine and temozolomide will cause an effect that is the sum of the effects individually exerted by the single components of the combination. In fact, administration of a combination of two or more components may give (in a way that cannot be foreseen a priori) the following:

(a) an enhanced effect, i.e. an effect which is greater than the sum of the effects produced by the single components administered individually (positive synergism),

(b) a null effect or an effect which is lower than that produced by individual administration of the single components (negative synergism), or

(c) an effect which is opposite to that exerted by individual administration of the components (inverse synergism). An opposite effect is meant to be, for example, an effect of stimulation of cell proliferation as opposed to an inhibitory effect.

The a priori unpredictability of the effect of any combination of a number of components, compared to the effects produced by the single components, is a fact that is universally recognized by the scientific community and that stands at the basis of the studies on synergism (which, as aforementioned, can be positive, negative or inverse). This is also testified by the fact that a simple bibliographical search made in the PubMed database (http://www.ncbi.nlm.nih.gov/pubmed/) by using “synergism” as a keyword will return as many as 68,501 documents, while a similar search made by using “positive synergism” as keywords will return as many as 3,474 documents, a similar search made by using “negative synergism” as keywords will return as many as 2,645 documents, and a similar search made by using “inverse synergism” as keywords will return 140 documents (searches updated to Jan. 24, 2016). The above considerations support the view according to which the huge number of documents about synergism in general suggests that this is a very common phenomenon when combinations of different molecules are used for studying their biological effects.

### EXAMPLE 3 (EXPERIMENTS BASED ON MIA PACA-2 CELL COUNT)

The MIA PaCa-2 cells were cultivated in T25 flasks and treated with the agents after reaching a confluence of 90%. After a 48 hours treatment, the cells were detached with trypsin-EDTA and suspended in medium consisting of 50% DMEM and 50% Trypan blue. The number of viable cells after each treatment was estimated by using the Neubauer counting chamber, excluding those cells which turned out positive to Trypan blue. At least three counts were made for each treatment.

The percentage of viable cells relative to the control was determined by the ratio between the number of treated cells and the number of untreated cells.

The following table shows the data from which the graph of FIG. 11 was derived.

In particular, Table 8 shows the data of berberine, gemcitabine and three combinations thereof (wherein the gemcitabine concentration was kept fixed, whereas the berberine concentration varied), applied to the MIA PaCa-2 pancreatic cancer cell line. The data were obtained via cell counting assay, from which the bar diagram of FIG. 11 was then obtained.

TABLE 8

| | Ber | Ber | Ber | Ber | Gemcitab | Gemcitab + | Gemcitab + | gencitab + |
|---|---|---|---|---|---|---|---|---|
| Untreated | 0.4 μM | 2.0 μM | 10 μM | 50 μM | 20 μM | Ber 0.4 μM | Ber 2.0 μM | Ber 10.0 μM |
| 100% | 88% | 51% | 33% | 24% | 33% | 15% | 18% | 13% |

The bar diagram of FIG. 11 shows the effects of treatments carried out on MIA PaCa-2 cells, indicating, in addition to the control, the percentage of viable cells after treatments with (a) gemcitabine administered individually at a concentration of 20 μM, (b) berberine administered individually at four different concentrations (0.4 μM, 2.0 μM, 10.0 μM and 50 μM), and (c) three binary combinations of 20 μM gemcitabine with berberine at three of the above-mentioned different concentrations (Combination I_{gem-ber}, containing 0.4 μM berberine—Combination II_{gem-ber}, containing 2.0 μM berberine—Combination III_{gem-ber}, containing 10.0 μM berberine).

The results show that berberine, when administered individually, produces, already at the 10 μM concentration, the same reduction in the percentage of viable cells as that produced by 20 μM gemcitabine (in both cases, after the two treatments the percentage of viable cells is reduced to a little more than 30%). This datum can be very interesting, if we consider that berberine belongs to food supplements, whereas gemcitabine implies, just like all other antitumour agents, inevitable and undesired toxicity.

However, the surprising fact is that the administration of combinations comprising 20 μM gemcitabine and berberine at concentrations in the range of 0.4 and 10 μM further reduces the number of viable cells to less than 20%.

Since the aim of the study described herein was to try to identify combinations comprising drugs already used for clinical purposes and alkaloids of C. majus which could provide advantageous therapeutic effects compared to the treatments known in the art, the observed effect due to the use of the three above-described combinations (I_{gem-ber}, II_{gem-ber} and III_{gem-ber}) seems to fully achieve the targets of the conducted research.

The use of combinations of Ukrain and gemcitabine in patients suffering from pancreatic cancer is known, but the employment of such an association has only been described in clinical use (in this respect, see Gansauge et al. [2007]). However, so far no study has been conducted, to the present Applicants' knowledge, which indicates the haematic concentrations of the individual components of Ukrain in patients treated with Ukrain or combinations thereof with gemcitabine.

Among the in vitro studies based on Ukrain associations, the study of Kurochkin et al. [2000] has been already mentioned above, which however relates to associations of Ukrain with etoposide, assayed in CHO cells.

Other in vitro studies on pancreatic cancer cell lines that involve Ukrain have been mentioned above. In this regard, it must be pointed out that Gagliano et al. [2012] describe in vitro studies on three pancreatic cancer cell lines (HPAF-II, HPAC and PL45) treated with Ukrain alone at concentrations of 5, 10 and 20 μM. This study provides important indications about the mechanism through which Ukrain performs its action, but it does not examine Ukrain in combination with other known molecules. On the same matter, it must also be pointed out that Funel et al. [2010] describe other in vitro studies on the same three pancreatic cancer cell lines (also in this case treated with 5, 10 and 20 μM Ukrain); also this study provides important information about the action mechanisms of Ukrain, without however examining the effects of Ukrain in combination with other known molecules.

Considering the fact that, at least in clinical use, combinations of Ukrain with gemcitabine have already been described for the treatment of pancreatic cancer, it is important herein to focus the attention on the results of an in vitro study, already mentioned above, that was carried out by the present Applicants in order to evaluate how the effects of binary combinations of gemcitabine with Ukrain or with each one of the three alkaloids of interest for the work described herein, may differ from one another.

Said study, the results of which are summarized in FIG. 12, demonstrates that the effect exerted on MIA PaCa-2 cells by a combination of Ukrain with gemcitabine is considerably different from the effects exerted by its individual components (berberine, chelidonine and protopine) used in binary combinations with gemcitabine. In fact, as already described, it can be observed that the combinations of Ukrain and gemcitabine produce a reduction in the dehydrogenase activity in MIA PaCa-2 cells that reaches approx. 50% when combinations are administered in which gemcitabine reaches a maximum concentration of 10 μM and Ukrain reaches a maximum concentration of 5%, expressed as volume/volume (v/v). A reduction of some interest in the dehydrogenase activity begins to be observed for combinations in which gemcitabine has a concentration starting from 10⁻⁵⁵ M and Ukrain has a concentration starting from 0.5% (v/v).

Within the concentration range wherein Ukrain appears in the binary combination with gemcitabine [5%-0.5% (v/v)], the three alkaloids of interest for the work described herein are approximately present at the following concentrations:

Berberine: approx. 0.2-0.02 μM

Chelidonine: approx. 4-0.4 μM

Protopine: approx. 160-16 μM

The binary combination of gemcitabine and berberine produces an effect of reduction in the dehydrogenase activity in MIA PaCa-2 cells ranging from approx. 75% to 25% when the (equimolar) concentrations of the two components vary in the range of 10^{-5.0} M [≡10 μM] to 10^{-5.5} M.

The binary combination of gemcitabine and chelidonine produces an effect of reduction in the dehydrogenase activity in MIA PaCa-2 cells that begins to become interesting (approx. 75%) only starting from concentrations close to 10 μM.

The binary combination of gemcitabine and protopine produces an effect, opposite to the desired one, of increasing the dehydrogenase activity in MIA PaCa-2 cells, with an irregular trend at all of the assayed concentrations.

Thus, in vitro (or in vivo) data relating to the use of combinations of Ukrain with gemcitabine cannot be considered to be anticipatory of the study described herein, because the effects of the binary combinations of the three alkaloids described herein with gemcitabine cannot be predicted on the basis of the effect of the binary combination of Ukrain with gemcitabine.

The dosages for a pharmaceutical product comprising the combination according to the invention can be obtained from the concentrations described in the examples; for this purpose, pharmaceutical products may be considered which include, in addition to the combination, also excipients for oral or injectable administration.

Such applications will still fall within the scope of the following claims.

Finally, for clarity's sake, all bibliographical references are listed below:
[Biswas et al 2008]
Biswas S J, Bhattacharjee N, Khuda-Bukhsh A R. (2008) Efficacy of a plant extract (Chelidonium majus L.) in combating induced hepatocarcinogenesis in mice. Food Chem Toxicol. 46(5):1474-87.
[Capistrano et al. 2015]
In vitro and in vivo investigations on the antitumour activity of Chelidonium majus. Capistrano I R, Wouters A, Lardon F, Gravekamp C, Apers S, Pieters L. Phytomedicine. 2015 Dec. 15; 22(14):1279-87. doi: 10.1016/j.phymed.2015.10.013. Epub 2015 Nov. 10. Work published after filing the patent application.
[Chaadaeva et al. 2009]
Chaadaeva A V, Tenkeeva I I, Moiseeva E V, Svirshchevskaia E V, Demushkin V P. (2009) Antitumor activity of the plant remedy peptide extract PE-PM in a new mouse T-lymphoma/eukemia model. Biomed Khim 55(1):81-8.
[Chen et al. 2012]
Chen C H, Liao C H, Chang Y L, Guh J H, Pan S L, Teng C M. Protopine, a novel microtubule-stabilizing agent, causes mitotic arrest and apoptotic cell death in human hormone-refractory prostate cancer cell lines. Cancer Lett. 2012 Feb. 1; 315(1):1-11. doi: 10.1016/j.canlet.2011.09.042.
[Chou, 2006]
Ting-Chao Chou, 2006: Theoretical Basis, Experimental Design, and Computerized Simulation of Synergism and Antagonism in Drug Combination Studies, Pharmacol Rev 58:621-681
[Chou, 2010]
Ting-Chao Chou, 2010: Ting-Chao Chou 2010: Drug Combination Studies and Their Synergy Quantification Using the Chou-Talalay Method, Cancer Res. 70:440-446)
[El-Readi et al 2013]
Modulation of multidrug resistance in cancer cells by chelidonine and Chelidonium majus alkaloids. El-Readi M Z, Eid S, Ashour M L, Tahrani A, Wink M. (2013) Phytomedicine 20(3-4):282-94.
[Fan et al. 2913]
Fan L X, Liu C M, Gao A H, Zhou Y B, Li J. Berberine combined with 2-deoxy-d-glucose synergistically enhances cancer cell proliferation inhibition via energy depletion and unfolded protein response disruption. Biochim Biophys Acta. 2013 November; 1830(11):5175-83. doi: 10.1016/j.bbagen.2013.07.010.
[Funel et al. 2010]
Ukrain affects pancreas cancer cell phenotype in vitro by targeting MMP-9 and intra-/extracellular SPARC expression. Funel N, Costa F, Pettinari L, Taddeo A, Sala A, Chiriva-Internati M, Cobos E, Colombo G, Milzani A, Campani D, Dalle-Donne I, Gagliano N. (2010) Pancreatology 10(5):545-52.
[Gagliano et al. 2012]
Pancreatic cancer cells retain the epithelial-related phenotype and modify mitotic spindle microtubules after the administration of ukrain in vitro. Gagliano N, Volpari T, Clerici M, Pettinari L, Barajon I, Portinaro N, Colombo G, Milzani A, Dalle-Donne I, Martinelli C. Anticancer Drugs. 2012 October; 23(9):935-46. doi: 10.1097/CAD.0b013e32835507bc.
[Gagliano et al. 2007]
Ukrain modulates glial fibrillary acidic protein, but not connexin 43 expression, and induces apoptosis in human cultured glioblastoma cells. Gagliano N, Moscheni C, Toni C, Donetti E, Magnani I, Costa F, Nowicky W, Gioia M. Anticancer Drugs. 2007 July; 18(6):669-76.
[Gansauge et al. 2002]
Gansauge F Ramadani M, Pressmar J, Gansauge S, Muehling B, Stecker K, Cammerer G, Leder G, Beger H G. 13.NSC-631570 (Ukrain) in the palliative treatment of pancreatic cancer. Results of a phase II trial. Langenbecks Arch Surg. 2002 March; 386(8):570-4. Epub 2002 February
[Gansauge et al. 2007] The clinical efficacy of adjuvant systemic chemotherapy with gemcitabine and NSC-631570 in advanced pancreatic cancer. Gansauge F, Ramadani M, Schwarz M, Beger H G, Lotspeich E, Poch B. Hepatogastroenterology. 2007 April-May; 54(75):917-20.
[Habermehl et al. 2006]
Proapoptotic activity of Ukrain is based on Chelidonium majus L. alkaloids and mediated via a mitochondrial death pathway. Habermehl D, Kammerer B, Handrick R, Eldh T, Gruber C, Cordes N, Daniel P T, Plasswilm L, Bamberg M, Belka C, Jendrossek V.BMC Cancer. 2006 Jan. 17; 6:14
[Jesionek et al. 2015
Investigation of the composition and antibacterial activity of Ukrain™ drug using liquid chromatography techniques. Jesionek W, Fornal E, Majer-Dziedzic B, Móricz Á M, Nowicky W, Choma I M., J Chromatogr A. 2016 Jan. 15; 1429, 340-347. doi: 10.1016/j.chroma.2015.12.015. Epub 2015 Dec. 8.
[Kurochkin et al. 2000]
Induction of apoptosis in cultured Chinese hamster ovary cells by Ukrain and its synergistic action with etoposide. Kurochkin S N, Kolobkov S L, Votrin I I, Voltchek I V. Drugs Exp Clin Res. 2000; 26(5-6):275-8.
[Lin et al. 2008]
Lin T H, Kuo H C, Chou F P, Lu F J.Berberine enhances inhibition of glioma tumor cell migration and invasiveness mediated by arsenic trioxide. BMC Cancer. 2008 Feb. 25; 8:58. doi: 10.1186/1471-2407-8-58.
[Liu et al. 2015]
Liu Q, Xu X, Zhao M, Wei Z, Li X, Zhang X, Liu Z, Gong Y, Shao C. Berberine induces senescence of human glioblastoma cells by downregulating the EGFR-MEK-ERK signaling pathway. Mol Cancer Ther. 2015 February; 14(2):355-63. doi: 10.1158/1535-7163.MCT-14-0634. Epub 2014 Dec. 12.
[Nadova et al. 2008]
Nadova S, Miadokova E, Alfoldiova L, Kopaskova M, Hasplova K, Hudecova A, Vaculcikova D, Gregan F, Cipak L. (2008) Potential antioxidant activity, cytotoxic and apoptosis-inducing effects of Chelidonium majus L. extract on leukemia cells. Neuro Endocrinol Lett. 29(5):649-52.
[Park et al. 2015]
Chelidonium majus L. extract induces apoptosis through caspase activity via MAPK-independent NF-κB signaling in human epidermoid carcinoma. A431 cells. Park S W, Kim S R, Kim Y, Lee J H, Woo H J, Yoon Y K, Kim Y I. (2015) Oncol Rep. 33(1):419-24. Epub 2014 Oct. 23
[Skivka et al. 2011]
The effect of monotherapy and combined therapy with NSC-631570 (ukrain) on growth of low- and high-metastasizing B16 melanoma in mice. Skivka L, Susak Y, Trompak O, Kudryavets Y, Bezdeneznikh N, Semesiuk N, Lykhova O. J Oncol Pharm Pract. 2011 December; 17(4):339-49. doi: 10.1177/1078155210382470. Epub 2010 Sep. 3.
[Staniszewski et al 1992]
Staniszewski A, Slesak B, Kolodziej J, Harlozińska-Szmyrka A, Nowicky J W. Lymphocyte subsets in patients with lung cancer treated with thiophosphoric acid alkaloid derivatives from Chelidonium majus L. (Ukrain). Drugs Exp Clin Res. 1992; 18 Suppl:63-7.
[Táborská et al. 1995]
Táborská E, Bochoráková H, Dostál J, Paulová H. [The greater celandine (Chelidonium majus L.)—review of present knowledge]. Ceska Slov Farm. 1995 April; 44(2):71-5. [Article in Czech])
[Voloshchuk et al. 2006]
Voloshchuk T. P., Patskovsky Y. V., Zayika L. A., Study of Ukrain composition using HPLC and UV-spectroscopy methods, Ukrainica Bioorganica Acta 2 (2006) 27-32

1. A composition for use in the treatment of cancer, comprising a pharmaceutically acceptable quantity of an agent having antineoplastic activity and of at least one alkaloid of Chelidonium majus.

2. The composition according to claim 1, wherein the alkaloid of Chelidonium majus is comprised in the group that includes: berberine, chelidonine, protopine, and combinations thereof.

3. The composition according to claim 1, wherein the agent having antineoplastic activity comprises at least one among gemcitabine and temozolomide or its MTIC metabolite.

4. The composition according to claim 3, wherein the concentration of the alkaloid is preferably comprised between 0.1 and 10 μM, whereas the concentration of temozolomide or its metabolite is lower than, or equal to, 20 μM.

5. The composition according to claim 4, wherein the alkaloid is berberine and its concentration is comprised between 0.464 and 10 μM.

6. The composition according to claim 4, wherein the alkaloid is chelidonine and its concentration is comprised between 0.4 and 8 μM.

7. The composition according to claim 4, wherein the alkaloid is protopine and its concentration is comprised between 0.4 and 8 μM.

8. The composition according to claim 3, wherein the alkaloid is berberine and its concentration is comprised between 0.4 and 10 μM, less than 2 μM, whereas the concentration of gemcitabine is preferably lower than or equal to 20 μM.

9. The composition according to claim 3, wherein the alkaloid is chelidonine and its concentration is comprised between 7 and 10 μM, less than 2 μM, whereas the concentration of gemcitabine is lower than or equal to 20 μM.

10. The composition according to claim 1, wherein the cancer to be treated is a pancreatic cancer when the antineoplastic agent is gemcitabine, or glioblastoma when the agent is temozolomide or its MTIC metabolite.

11. (canceled)

12. A pharmaceutical product, comprising a composition including a pharmaceutically acceptable quantity of an agent having antineoplastic activity of at least one alkaloid of Chelidonium majus, temozolomide, an alkaloid, and excipients for oral or injectable administration.
